(19) United States
(12) Patent Application Publication
(10) Pub. No.: US 2026/0053518 A1
(43) Pub. Date: Feb. 26, 2026

(54) SYSTEMS, DEVICES AND METHODS FOR IVL PROCEDURES WITH IMPROVED EFFICIENCY, EFFECTIVENESS AND SAFETY

(71) Applicant: Cardiovascular Systems, Inc., Santa Clara, CA (US)

(72) Inventors: Austin P. Petronack, Plymouth, MN (US); Scott P. Boeshart, Plymouth, MN (US); Jeffrey T. Moriarty, Roseville, MN (US)

(21) Appl. No.: 19/307,955

(22) Filed: Aug. 22, 2025

Related U.S. Application Data
(60) Provisional application No. 63/686,885, filed on Aug. 26, 2024.

(51) Int. Cl. A61B 17/22; A61B 17/00

(52) U.S. Cl. CPC A61B 17/22022; A61B 2017/0003; A61B 2017/22051

(57) ABSTRACT

A device may include an elongate member; a fluid-fillable enclosure formed of a material and surrounding a distal region of the elongate member; at least one pair of spaced-apart electrodes operatively associated with the elongate member and within the fluid-fillable enclosure. Each pair of the at least one pair of spaced-apart electrodes form a spark gap. A voltage pulse generator is in operative electrical communication and association with the at least one pair of spaced-apart electrodes. A controller in operative association with the voltage pulse generator and configured to control the voltage pulse generator. The device is configured to produce a plurality of electrical arcs across the at least one pair of spaced-apart electrodes, each of the plurality of electrical arcs being produced in response to a voltage pulse provided by the voltage pulse generator. The device is further configured to generate at least one non-arcing voltage pulse.

### CROSS-REFERENCE TO RELATED APPLICATIONS

This application claims the benefit of and priority to U.S. Provisional Patent Application Ser. No. 63/686,885 filed on Aug. 26, 2024 and entitled “SYSTEMS, DEVICES AND METHODS FOR IVL PROCEDURES WITH IMPROVED EFFICIENCY, EFFECTIVENESS AND SAFETY” and which application is expressly incorporated herein by reference in its entirety.

### BACKGROUND

### Background and Relevant Art

A variety of techniques and instruments have been developed for use in the removal or repair of tissue in arteries and similar body passageways, including removal and/or cracking of calcified lesions within the passageway and/or formed within the wall defining the passageway. A frequent objective of such techniques and instruments is the removal of atherosclerotic plaque in a patient's arteries. Atherosclerosis is characterized by the buildup of fatty deposits (atheromas) in the intimal layer (i.e., under the endothelium) of a patient's blood vessels. Very often over time what initially is deposited as relatively soft, cholesterol-rich atheromatous material hardens into a calcified atherosclerotic plaque, often within the vessel wall. Such atheromas restrict the flow of blood, cause the vessel to be less compliant than the same vessel absent atheromas, and therefore often are referred to as stenotic lesions or stenoses, the blocking material being referred to as stenotic material. If left untreated, such stenoses can cause angina, hypertension, myocardial infarction, strokes and the like.

Angioplasty, or balloon angioplasty, is an endovascular procedure to treat by widening narrowed or obstructed arteries or veins, typically to treat arterial atherosclerosis. A collapsed balloon is typically passed through a pre-positioned catheter and over a guide wire into the narrowed occlusion and then inflated to a fixed pressure. The balloon forces expansion of the occlusion within the vessel and the surrounding muscular wall until the occlusion yields from the radial force applied by the expanding balloon, opening up the blood vessel with an inner diameter that is similar to the native vessel in the occlusion area and, thereby, improving blood flow.

Balloon angioplasty can be enhanced by using intravascular lithotripsy (IVL). Generally, known IVL devices include a voltage pulse generator in operative communication with one or more pairs of electrodes mounted on a catheter and within an inflatable balloon of a catheter. The balloon is inflated using a conductive fluid. One or a series of high-voltage pulses can be applied to the pairs of electrodes causing arcing, resulting in pressure waves that travel from the pairs of electrodes, through the fluid, and to the lesions, helping to crack or break up the lesions to restore compliance.

Intravascular lithotripsy systems, devices and methods have been described by Applicant. See PCT/2022/074607, filed by Applicant on Aug. 5, 2022 and entitled “INTRAVASCULAR LITHOTRIPSY BALLOON SYSTEMS, DEVICES AND METHODS”, and PCT/US2023/085868, filed by Applicant on Dec. 23, 2023 and entitled “INTRAVASCULAR LITHOTRIPSY SYSTEM WITH IMPROVED DURABILITY, EFFICIENCY AND PRESSURE OUTPUT VARIABILITY”, the entire contents of each of which are hereby incorporated by reference.

The production of electrical arcs may result in degradation or erosion of the conductive material of the electrodes which may reduce efficiency of the production of the electrical arcs overtime. Further, the conductivity of the conductive fluid within the balloon may be altered by the electrical arcs which may also contribute to reduced efficiency of the electrical arcs over time. In addition, heat buildup may occur. The net result of the procession of voltage pulses producing electrical arcs is at least a reduction of therapeutic efficiency, and in some cases may contribute to elevating the risk of serious failures such as balloon rupture.

Currently known IVL systems require an integrated periodic deflation and reinflation of the balloon between pulse delivery cycles, which serves to dissipate heat and remove residual gas bubbles through fluid evacuation from the balloon while allowing tissue reperfusion to mitigate ischemia. These periodic balloon deflations are suggested in instructions for use to be executed at predetermined intervals or times and, when performed, are executed manually without regard to the actual gas concentration or buildup within the balloon. Because the known processes rely entirely on the operator to stop IVL treatment to degas the balloon at the suggested time intervals, errors may be introduced which lead to reduction in therapeutic efficiencies. Other known IVL systems force an operator to pause for 10 seconds after a predetermined number of voltage pulses have been generated. None of the known IVL systems provide a means for confirming that an IVL device is in need of degassing or confirming that the degassing process was sufficient.

The subject matter claimed herein is not limited to embodiments that solve any disadvantages or that operate only in environments such as those described above. Rather, this background is only provided to illustrate one exemplary technology area where some embodiments described herein may be practiced.

### BRIEF SUMMARY

In some aspects, the techniques described herein relate to an intravascular lithotripsy (“IVL”) catheter with improved efficiency, including: an elongate member; a fluid-fillable enclosure formed of a material and surrounding a distal region of the elongate member; at least one pair of spaced-apart electrodes operatively associated with the elongate member and within the fluid-fillable enclosure, wherein each pair of the at least one pair of spaced-apart electrodes form a spark gap; a voltage pulse generator in operative electrical communication and association with the at least one pair of spaced-apart electrodes; and a controller in operative association with the voltage pulse generator and configured to control the voltage pulse generator, wherein the IVL system is configured to produce a plurality of electrical arcs across the at least one pair of spaced-apart electrodes, each of the plurality of electrical arcs being produced in response to a voltage pulse provided by the voltage pulse generator, and wherein the IVL system is further configured to generate at least one non-arcing voltage pulse.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

Additional features and advantages will be set forth in the description which follows, and in part will be obvious from the description, or may be learned by the practice of the teachings herein. Features and advantages of the invention may be realized and obtained by means of the instruments and combinations particularly pointed out in the appended claims. Features of the present invention will become more fully apparent from the following description and appended claims, or may be learned by the practice of the invention as set forth hereinafter.

### BRIEF DESCRIPTION OF THE DRAWINGS

In order to describe the manner in which the above-recited and other advantages and features can be obtained, a more particular description of the subject matter briefly described above will be rendered by reference to specific embodiments which are illustrated in the appended drawings. Understanding that these drawings depict only typical embodiments and are not therefore to be considered to be limiting in scope, embodiments will be described and explained with additional specificity and detail through the use of the accompanying drawings in which:

FIG. 1 illustrates a schematic view of an IVL system.

FIG. 2 illustrates a schematic view of part of an IVL system.

FIG. 3 illustrates a graphic illustration of a typical timing of applied voltage and current during application of voltage and generation of an electrical arc between spaced-apart electrodes.

FIG. 4 illustrates an exemplary arcing waveform for one embodiment of the current disclosure.

FIG. 5 illustrates an exemplary non-arcing waveform for one embodiment of the current disclosure.

FIG. 6 illustrates an exemplary non-arcing waveform for one embodiment of the current disclosure.

FIG. 7 illustrates a comparison of the exemplary waveforms of FIGS. 4-6.

FIG. 8 illustrates one embodiment of a flow diagram of the present disclosure.

### DETAILED DESCRIPTION

Some embodiments illustrated herein introduce at least one non-arcing voltage pulse during execution of an IVL procedure.

A voltage pulse that is designed to not induce an electrical arc between spaced-apart electrodes may comprise a magnitude of, e.g., 1,200V or less, or 1000V or less, but these magnitudes are exemplary only. Alternatively, a shorter duration of voltage pulse generation may be employed, as compared with voltage pulses designed to generate electrical arcs. For example, the non-arcing voltage pulse may comprise a duration of one to hundreds of microseconds such as, without limitation a duration up to about 250 microseconds. In other embodiments, a non-arcing voltage pulse may comprise a duration of application that may exceed 250 microseconds and the voltage may comprise more than 1,200V. As will be illustrated further below, one embodiment of a non-arcing voltage pulse may comprise a voltage of, e.g., 1,200V or less coupled with a duration of application of voltage to spaced-apart electrodes of an exemplary IVL system in the range of about one to about 250 microseconds. The voltage pulse is configured to not produce an electrical arc or spark event across spaced-apart electrodes in an IVL system, and is hereinafter referred to as a “non-arcing voltage pulse”. Thus in some embodiments, such a non-arcing voltage pulse may not produce a visible electrical arc between spaced-apart electrodes and, therefore does not produce the resultant gas and heat formation associated with electrical arc production.

In contrast, an arcing voltage pulse may comprise a magnitude between about 2000V and about 4000V, with a duration up to about 25 microseconds. In some embodiments, a non-arcing voltage pulse may produce no cavitation bubbles. In some embodiments, a non-arcing voltage pulse may produce cavitation bubbles but at a far slower rate than an arc-inducing voltage pulse. In some embodiments, a non-arcing voltage pulse may produce smaller bubbles than an arc-inducing voltage pulse. In some embodiments, the non-arcing voltage pulse may not even produce more than a de minimus bubble formation.

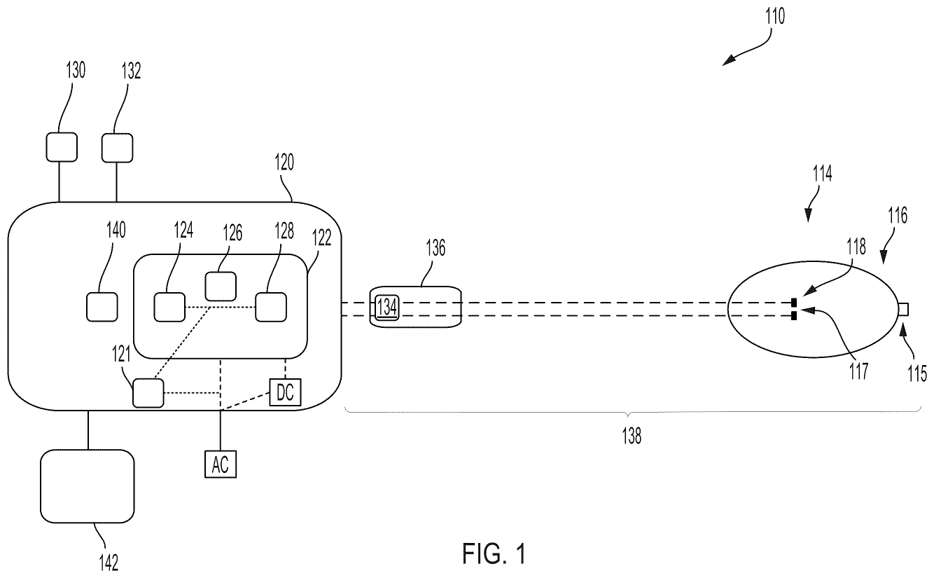

As illustrated in FIG. 1, a diagrammatic or schematic layout of portions of an exemplary IVL system 110 is provided. The illustrative IVL system 110 comprises a catheter assembly 114 including an elongate body, embodied as a catheter having guidewire 115, and a fluid-filled member 116 configured to contain conductive fluid therein, exemplified by an inflatable balloon, disposed near the distal end of the elongate body and arranged to receive fluid for inflation to facilitate IVL therapy. A set of dischargeable spaced apart electrodes 118 are shown arranged within the exemplary fluid-filled member 116, at least some of which are spaced apart by a gap 117 from each other to create a spark or electrical arc between the set of spaced-apart electrodes 118.

The IVL system embodiments described herein may be used in connection with electrodes that are within a fluid-filled member 116 configured to contain a fluid, e.g., a conductive fluid, therein. The fluid-filled member 116 embodiments may include an inflatable balloon or enclosure as shown in FIG. 1, which may be compliant or non-compliant and serves to contain the fluid such that the spaced-apart electrodes 118 are preferably fully submerged within the contained fluid. In addition, the fluid-filled member 116 may comprise a fillable member that is at least partially rigid and/or not flexible. In other embodiments, the fluid-filled member 116 may contain the fluid therein and wherein the set of spaced apart electrodes 118 are most preferably fully submerged within the contained fluid during generation of an electrical arc between the spaced-apart electrodes during an IVL procedure.

Alternatively, the IVL system control embodiments of the present disclosure may be used in connection with electrodes that are not located or surrounded by a fluid-filled or fillable member 116. In these embodiments, the IVL system may comprise one or more sets of spaced-apart electrodes 118 that may be continuously or periodically exposed to saline or other fluid and, during the exposure, the IVL system may generate an electrical arc between the spaced-apart electrodes 118.

Each of the spaced-apart electrodes in FIG. 1 is arranged in communication (as suggested by dashed line conductors) with an electric pulse generation system, or voltage pulse generator 120 to receive high voltage electrical energy for spark generation to create pressure waves for IVL therapy. In the illustrative embodiment, one electrode may be grounded and the other provided with high voltage from the voltage pulse generator 120, although in some embodiments, any voltage differential may be applied. The voltage pulse generator 120 may include a capacitor bank 121, an IVL control system 122 comprising a processor 124 configured for executing instructions stored on memory 126 and communications signals via circuitry 128 for IVL operations according to the processor governance. The capacitor bank 121, processor 124, memory 126, and circuitry 128 may be arranged in communication with each other (as suggested via dashed lines) to facilitate disclosed operations. In some embodiments, the instructions stored on memory 126 include instructions to configure the processor 124 to cause a non-arcing pulse based on certain criteria as will be discussed in more detail below.

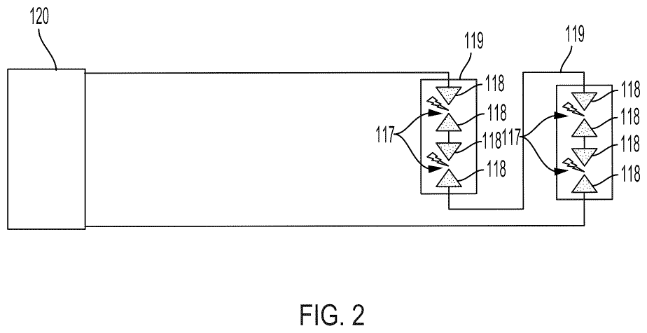

An exemplary IVL system is shown in FIG. 2, illustrating one method for applying voltage pulses to the system, resulting in current flow through the exemplary system to each set of spaced-apart electrodes 118, where each set of spaced-apart electrodes 118 forms a spark gap 117. In the illustrated example, two sets of spaced apart electrodes form an emitter 119. A given emitter 119 may include two spark gaps so as to accomplish more complete energy coverage, such as when the spark gaps are located on conductive bands around a catheter lumen to allow for pressure waves in multiple directions. Note that in other embodiments, emitters may include a single spark gap or more than two spark gaps. The emitters 119, and thus the four sets of spaced-apart electrodes (and thus spark gaps 117) are connected in series. FIG. 2 illustrates two emitters 119 defined within a support body. Application of a voltage pulse of sufficient magnitude and/or duration from the voltage pulse generator 120 to the sets of serially connected spark gaps 117 will result in a production of electrical arcs as follows: a first arc across a first spark gap; a second arc across a second spark gap, a third arc across a third spark gap and a fourth arc across a fourth spark gap.

Appropriate control of such high-energy systems can also require achieving sufficient energy at the discharge site to create an electrical arc across the one or more spark gaps of an IVL system. Embodiments of the IVL systems, devices and methods described within the present disclosure may include operation for adjusting the total electrical energy provided to the system at each emitter. Such control systems for intravascular lithotripsy systems, devices and methods have been described by Applicant. See PCT/2023/079209, filed by Applicant on Nov. 9, 2023 and entitled “CONTROL OF IVL SYSTEMS, DEVICES AND METHODS THEREOF”; U.S. Ser. No. 18/290,173, filed by Applicant on Nov. 10, 2023 and entitled “CONTROL OF IVL SYSTEMS, DEVICES AND METHODS THEREOF”; U.S. Ser. No. 18/506,339, filed by Applicant on Nov. 10, 2023 and entitled “CONTROL OF IVL SYSTEMS, DEVICES AND METHODS THEREOF”; U.S. Ser. No. 18/506,416, filed by Applicant on Nov. 10, 2023 and entitled “CONTROL OF IVL SYSTEMS, DEVICES AND METHODS THEREOF”; and U.S. Ser. No. 18/506,428, filed by Applicant on Nov. 10, 2023 and entitled “CONTROL OF IVL SYSTEMS, DEVICES AND METHODS THEREOF”, the entire contents of which are hereby incorporated by reference. The voltage pulse may be generated by a discharging energy stored in the voltage pulse generator 120, and in particular, in some embodiments, discharging energy stored in the voltage pulse generator 120, including potentially the capacitor bank 121 (FIG. 1) which may produce an electrical arc across a spark gap between two spaced-apart electrodes. In some embodiments, the voltage pulses may be generated in one or more series of voltage pulses, wherein each adjacent series of voltage pulses may be spaced-apart or separated by time or other mechanism. As noted previously, in some embodiments a voltage pulse may be provided that intentionally does not result in arcing across the spark gaps 117.

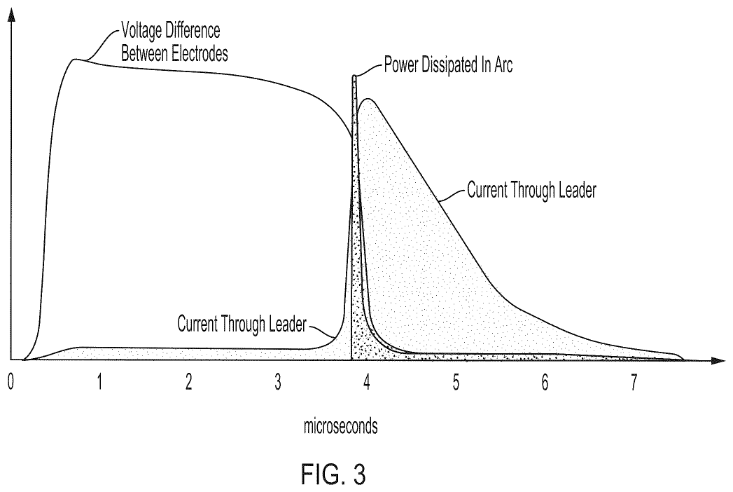

FIG. 3 graphically illustrates the drop in impedance of a current leader across a spark in an IVL system such as shown in FIGS. 1 and/or 2 following application of a single voltage pulse to one of two spaced-apart electrodes as the current leader develops into an electrical arc between the spaced-apart electrodes. This, in turn, causes the power dissipated in the electrical arc to peak sharply while the voltage and current between the electrodes are both relatively high. The current reaches a peak and the voltage drops, both very rapidly, indicating that an electrical arc between the spaced-apart electrodes is present, or has occurred. The peak of the power dissipated in the electrical arc indicates the relatively short time interval during which all the useful work of heating the growing leader into an arc is performed. The graphic illustration of FIG. 3 is exemplary of one aspect of an IVL procedure that is configured to produce or generate pressure waves.

As noted previously, IVL systems and devices may include a voltage pulse generator 120, including potentially the capacitor bank 121 which is triggered to release energy to generate electrical arcs. Following production of a series of voltage pulses, each of which is designed to generate an electrical arc as described above, the capacitor bank may retain residual energy in the form of voltage which may present a risk to the user/operator and/or the patient.

Further, IVL procedures result in degradation of the spaced-apart electrodes 118 during the electrical arcing process. In addition, the conductivity of the surrounding fluid within the balloon or enclosure may be affected by the procession of electrical arcs generated during an IVL procedure, for example, due to gas bubbles and/or contaminants being formed in the fluid. Embodiments illustrated herein include Monitoring and/or estimation of the progression of these parameters during an IVL procedure using a non-arcing pulse as described further herein.

In addition, tracking the number of voltage pulses generated during a series of voltage pulses, as well as the overall number of voltage pulses generated across a plurality of series of voltage pulses is a monitored parameter. Generally, this is achieved in known systems by simply counting the generated voltage pulses, wherein each voltage pulse is configured to produce an electrical arc.

Moreover, gas generation is an issue with IVL systems and devices that make use of an enclosure such as an exemplary balloon and one or more sets spaced-apart electrodes defining a spark gap, or emitters, surrounded by a conductive fluid held within the enclosure. Gas is generated as the fluid within the enclosure is vaporized during each electrical arc event and may accumulate following a series of voltage pulses, wherein each voltage pulse generates an electrical arc event. In some embodiments, each voltage pulse in a series of voltage pulses may be consecutive and may be produced in succession. The gas buildup within the balloon or enclosure is undesirable as it may, among other things, attenuate the pressure pulse or wave generated by the spaced-apart electrodes or emitters before the energy strikes a target treatment site, e.g., an occlusion and/or calcification within a blood vessel's wall. Further, the gas buildup may degrade the efficiency of formation of the electrical arc or spark across the spark gap of the spaced-apart electrodes or emitters. Systems, Devices and Methods for detection of gas buildup have been described in U.S. application 63/642,019 filed by Applicant on May 3, 2024 and entitled SYSTEMS, DEVICES AND METHODS FOR DETECTION OF IVL GAS BUILDUP, the entire contents of which are hereby incorporated by reference.

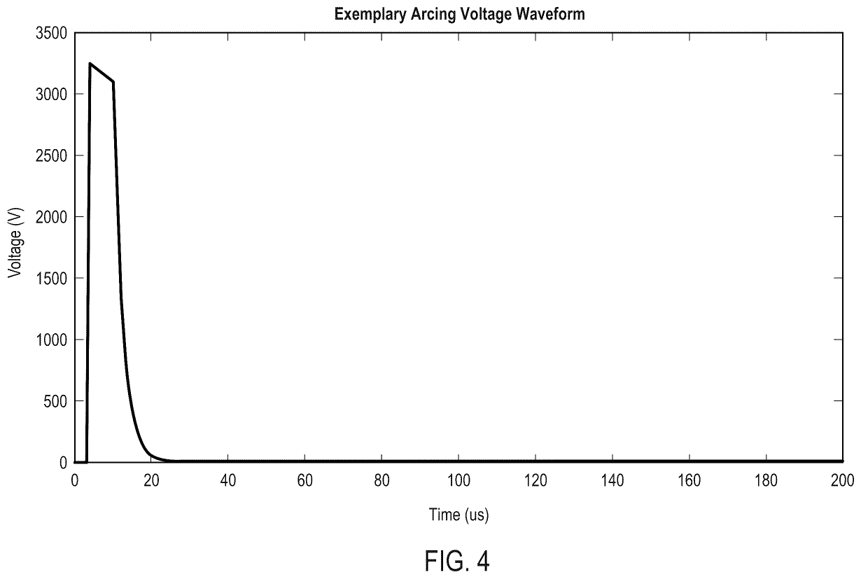

FIG. 4 illustrates an exemplary arcing voltage waveform generated by an exemplary IVL system as described herein, with applied voltage of approximately 3250V across the spark gaps 117 and a time duration of voltage application to spaced-apart electrodes 118 of approximately 7 microseconds.

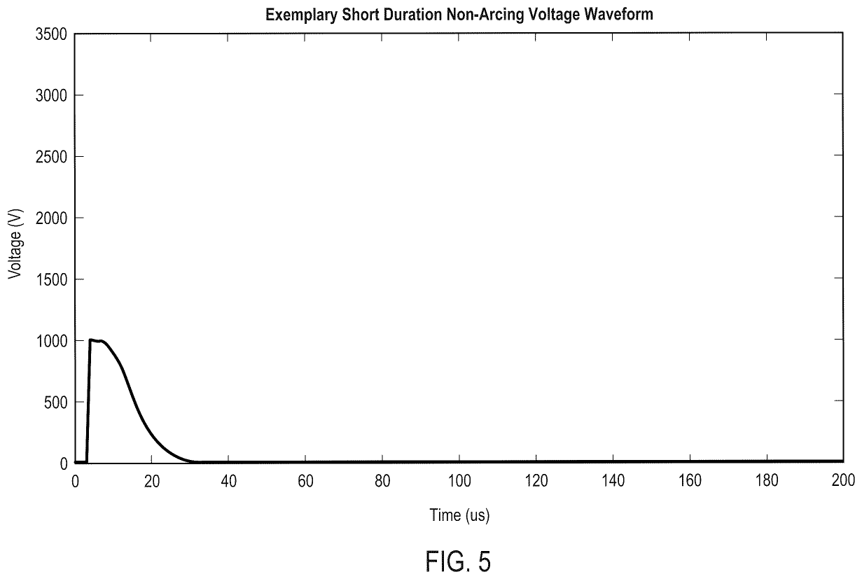

FIG. 5 illustrates an exemplary non-arcing voltage waveform generated by an embodiment IVL system, with an applied voltage of approximately 1000V and an exemplary duration of voltage application to spaced-apart electrodes 118 of the IVL system of approximately 5-7 microseconds.

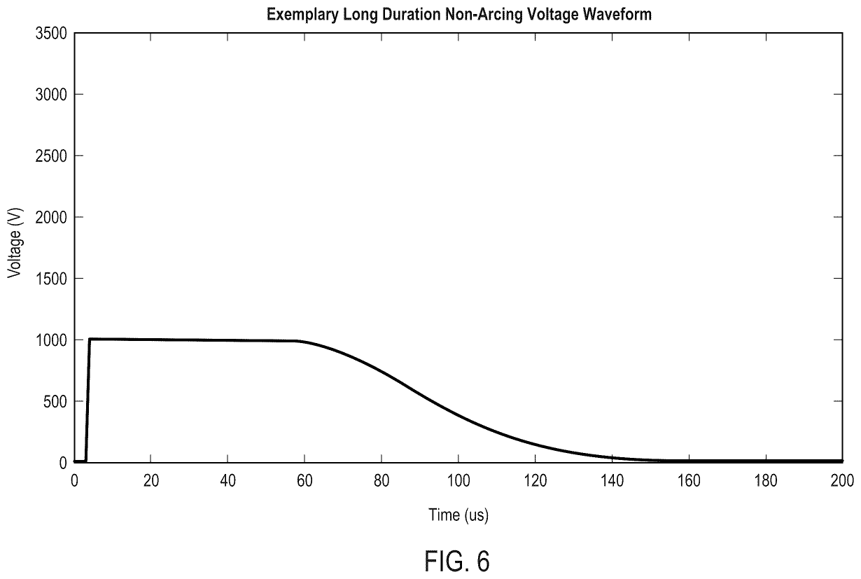

FIG. 6 illustrates an exemplary non-arcing voltage waveform generated by an embodiment IVL system, with an applied voltage of approximately 1000V and an exemplary duration of voltage application to spaced-apart electrodes of the IVL system of approximately 60 microseconds. Thus, the non-arcing voltage waveform of FIG. 6 is generated by a longer duration of voltage application to the spaced-apart electrodes 118 than the waveform of FIG. 5.

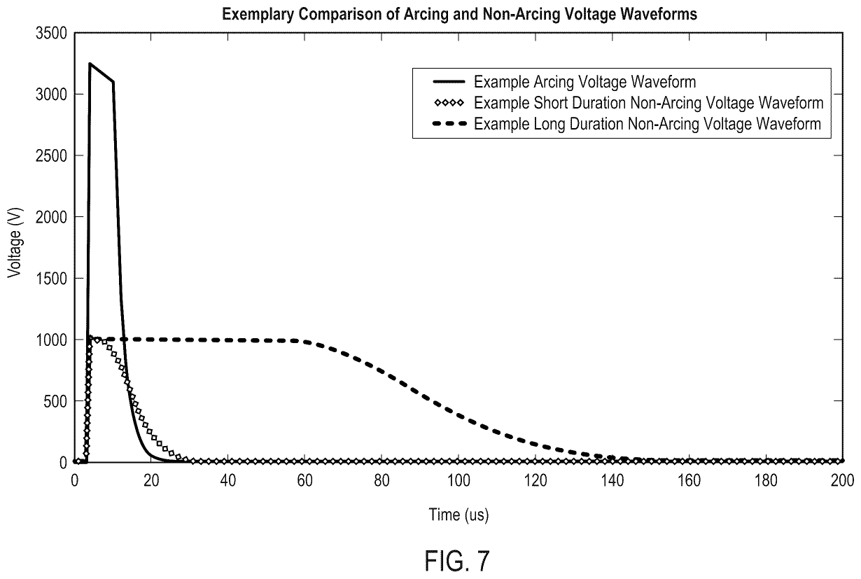

FIG. 7 provides an overlaid comparison of the waveforms shown in FIGS. 4-6 to further illustrate the differences between arcing and non-arcing waveforms.

As shown in FIG. 1 and with continued reference to FIG. 2, the exemplary IVL system 110 is provided comprising an EPROM 134, which is discussed further below. The EPROM 134 is in operative communication and association with the processor 124, which is in the illustrated embodiment part of the voltage pulse generator 120. In other embodiments, the IVL control system 122, and associated processor 124, memory 126 and related circuitry 128 may be in operative and electrical communication with the voltage pulse generator 120, but located in a different physical location than the voltage pulse generator 120. The EPROM 134 is illustrated as located within or in operative association with the handle 136 of a catheter 138, which is in operative association with the fluid-filled member 116.

The processor 124 is in operative and/or electrical association with a voltage monitor 40 that is illustrated as located within the voltage pulse generator 120. In some embodiments, the voltage monitor may be located in a separate location apart from the voltage pulse generator 120. The voltage pulse generator 120 may be in operative and electrical association with one or more sets of spaced-apart electrodes 118. As illustrated, the various spaced-apart electrodes are connected in a serial electrical connection with the voltage pulse generator 120 operatively and/or electrically connected with a more proximally located emitter 119. Alternative connection associations between the set(s) of spaced-apart electrodes 118 or emitter(s) 119 and the voltage pulse generator 120 may be provided in alternative embodiments. For example, the two sets of spaced-apart electrodes may be connected in a parallel arrangement. Further, the voltage pulse generator 120 may be operatively and/or electrically associated with a more distal spaced-apart set of electrode 118 or emitter 119. In addition, a voltage monitor 130 and/or a current monitor 132 may be provided in operative connection and communication with the IVL control system 122 and/or the voltage pulse generator 120. Finally, a display may be operatively and/or electrically associated with the IVL control system 122.

Some embodiments may comprise a predetermined minimum time parameter and a minimum voltage parameter, that when applied result in a threshold parameter minimum voltage dissipation threshold which may comprise a voltage magnitude or a voltage dissipation rate. These predetermined parameters and threshold may be programmed into the EPROM (erasable programmable read-only memory) 134 as well understood in the art. In some embodiments, the EPROM may be disposed within a handle 136 of the catheter 138. This arrangement in some embodiments allows the predetermined minimum time and voltage parameters, and the resultant minimum voltage dissipation threshold to be programmed specifically to a certain model and/or device and/or system. Alternatively, the predetermined parameters and threshold may be stored within the memory 126 and/or processor 124.

Consequently, production of a non-arcing voltage pulse relies on generation of a predetermined non-arcing voltage magnitude and/or a predetermined non-arcing duration of application of a predetermined voltage magnitude to the spaced-apart electrodes. These parameters may be programmed into the EPROM 134 or, as discussed above, stored within the memory 126 and/or processor 124. When the non-arcing parameters are executed by the IVL system, the result will provide a non-arcing voltage pulse.

The exemplary system 110 of FIG. 1 comprises a capacitor bank that may be energized for programmed energy release to initiate voltage pulses. As noted, the voltage pulses may be arranged in a series of voltage pulses and may comprise at least one voltage pulse configured to generate electrical arcs between spaced-apart electrodes. At the conclusion of the generation of the arc-inducing or producing voltage pulses, the voltage pulse generator 120, including potentially the capacitor bank 121 may retain residual energy that may be of a magnitude that presents a risk to the operator of the IVL system and/or to the patient. Including a non-arcing voltage pulse reduces the residual energy of the voltage pulse generator 120, including potentially the capacitor bank 121 and reduces the resultant risk.

The non-arcing pulse may be programmed into the IVL system 110 as a required operation following one or more voltage pulses and/or a series of voltage pulses, wherein each voltage pulse is of a magnitude and/or duration designed to produce an electrical arc.

Alternatively, the non-arcing pulse may be executed following a measurement or monitoring of the level of residual energy remaining in the voltage pulse generator 120, including potentially the capacitor bank 121. In this embodiment, a non-arcing voltage pulse may be required, or recommended, only if the measured or monitored residual energy of the voltage pulse generator 120, including potentially the capacitor bank 121 is determined to be above a predetermined threshold. Production of the non-arcing voltage pulse in this embodiment may be executed automatically according to programmed instructions to reduce the residual energy of the capacitor bank. Alternatively, a determination that the residual energy of the capacitor bank is above the threshold may be used to alert the operator through the display or other means by displaying, or otherwise annunciating, that the residual energy of the capacitor bank is over the predetermined threshold and should be reduced by executing a non-arcing voltage pulse.

In some embodiments, a non-arcing voltage pulse may be detected or measured by measuring voltage with a voltage sensor at the output of the voltage pulse generator 120, including potentially the capacitor bank 121 during the discharge. In other embodiments, a non-arcing voltage pulse may be detected or measured by measuring voltages at the voltage pulse generator 120, including potentially the capacitor bank 121 before voltage discharge and after voltage discharge. In some embodiments, a non-arcing voltage pulse may be detected by monitoring and/or determining the duration of voltage pulse application which will be less than a predetermined threshold duration. In some embodiments, a non-arcing voltage pulse may be detected by monitoring the resultant waveform of the voltage pulse and comparing it with a known waveform produced by an arc-inducing voltage pulse.

In certain embodiments, the residual energy may continue to be above the predetermined threshold following execution of one non-arcing voltage pulse. In this case, the programmed instructions may automatically execute at least one more non-arcing voltage pulse to reduce the capacitor bank residual energy below the predetermined threshold. Alternatively, the operator may be alerted to consider executing another non-arcing voltage pulse.

In certain embodiments, upon determination of a residual energy level of the capacitor bank is over the predetermined threshold, the processor 124 may be configured to lock out, or prevent, any further generation of voltage pulses of sufficient magnitude to generate electrical arcs until the residual energy of the capacitor bank is determined to be below the predetermined threshold.

In certain embodiments, the reduction residual energy within the capacitor bank may comprise a closing of gates and/or relays to reduce the residual energy by release. In these embodiments, the non-arcing voltage pulse comprises simply releasing at least some of the residual energy from the capacitor bank to the emitter(s).

With continued reference to FIGS. 1-7, execution of a non-arcing voltage pulse that is interposed within, or between, two or more arc-inducing voltage pulses provides an alternative mechanism for counting the generated voltage pulses designed to induce or produce an electrical arc between the spaced-apart electrodes.

As briefly noted above, IVL procedures result in degradation of the spaced-apart electrodes during the electrical arcing process. In addition, the conductivity of the surrounding fluid within the balloon or enclosure may be affected by the procession of electrical arcs generated during an IVL procedure. Monitoring and/or estimation of the progression of these parameters during an IVL procedure would be very advantageous. Thus, as discussed above, a non-arcing voltage pulse may be delivered following generation of a plurality of voltage pulses designed to produce electrical arcs. The number of arc-inducing voltage pulses generated before generation of a non-arcing voltage pulse may be programmed into the processor 124 and/or memory 126, and/or the EPROM 134. The generation of the non-arcing voltage pulse may, as described above, be programmed and thus automatically generated, or may result in an alert to the operator to generate a non-arcing voltage pulse.

The following discussion now refers to a number of methods and method acts that may be performed. Although the method acts may be discussed in a certain order or illustrated in a flow chart as occurring in a particular order, no particular ordering is required unless specifically stated, or required because an act is dependent on another act being completed prior to the act being performed.

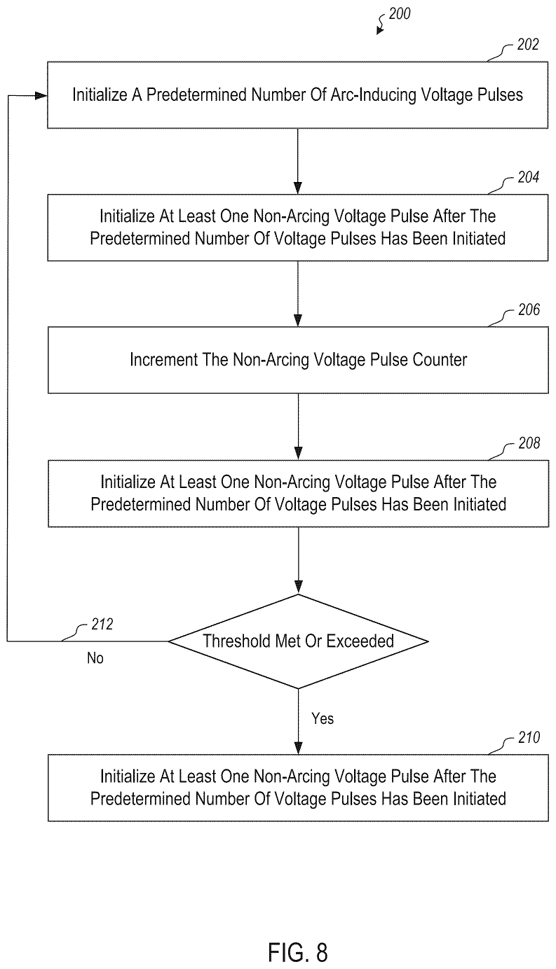

An exemplary flow diagram for one method of the current disclosure is provided in FIG. 8.

Thus, FIG. 8 illustrates an exemplary method 200 for counting voltage pulses. Step 202 comprises initiating a predetermined number of arc-inducing voltage pulses as described above. Step 204 comprises initiating at least one non-arc inducing voltage pulse, also as described above, after the predetermined number of arc-inducing voltage pulses has been reached. Step 206 comprises incrementing a non-arcing voltage pulse counter which may be located within the EPROM 134, IVL control system 122, and/or processor 124 and which is configured to determine the number of initiated arc-inducing voltage pulses based on the number of initiated arc-inducing based on the number non-arc inducing voltage pulses. Step 208 comprises comparing the determined number of initiated arc-inducing voltage pulses with a threshold number of arc-inducing voltage pulses. Step 210 comprises determining whether the threshold number has been met or exceeded and, if exceeded then stopping or preventing or locking out any additional arc-inducing voltage pulses. Step 212 comprises determining if the predetermined threshold number has not been met or exceeded, then return to step 202.

Generation and delivery of a non-arcing voltage pulse, with concurrent monitoring of voltage, via the voltage monitor 130 or equivalent, and/or current, via the current monitor 132 or equivalent, also enables estimation of system impedance. A determination of system impedance that is greater than a predetermined programmed threshold may indicate that the electrode material degradation has exceeded an acceptable level, which may also be referred to as a level of efficiency or effectiveness of electrical arc production. In addition, a determination that the system impedance is greater than the predetermined threshold may indicate that the conductivity of the conductive fluid within the balloon or enclosure has decreased below an acceptable level.

The determination that either the electrode material degradation or the conductive fluid's conductivity are outside the acceptable, efficient or effective level may be used to modify subsequent arc-inducing voltage pulses by adjusting, e.g., increasing at predetermined increment (s), the voltage magnitude and/or duration of the voltage pulse applied to the spaced-apart electrodes to accommodate, and adapt to, the changing impedance conditions.

Alternatively, determination that the electrode material degradation and/or the conductive fluid conductivity are outside the acceptable, efficient or effective level may result in the processor 124 locking out further arc-inducing voltage pulses until the situation is mitigated or corrected. In this latter embodiment, a second predetermined threshold may be provided that, if exceeded, results in such a lockout.

Accordingly, certain embodiments of the disclosure require initiating a non-arcing voltage pulse that is executed after a series (in some embodiments a predetermined number) of arc-inducing voltage pulses is initiated and completed. Certain embodiments do not include the gas buildup detection described herein.

The description of the invention and its applications as set forth herein is illustrative and is not intended to limit the scope of the invention. Features of various embodiments may be combined with other embodiments within the contemplation of this invention. Variations and modifications of the embodiments disclosed herein are possible, and practical alternatives to and equivalents of the various elements of the embodiments would be understood to those of ordinary skill in the art upon study of this patent document. These and other variations and modifications of the embodiments disclosed herein may be made without departing from the scope and spirit of the invention.

The following illustrates one or more embodiments.

Clause 1. An intravascular lithotripsy (“IVL”) catheter with improved efficiency, comprising: an elongate member; a fluid-fillable enclosure formed of a material and surrounding a distal region of the elongate member; at least one pair of spaced-apart electrodes operatively associated with the elongate member and within the fluid-fillable enclosure, wherein each pair of the at least one pair of spaced-apart electrodes form a spark gap; a voltage pulse generator in operative electrical communication and association with the at least one pair of spaced-apart electrodes; and a controller in operative association with the voltage pulse generator and configured to control the voltage pulse generator, wherein the IVL system is configured to produce a plurality of electrical arcs across the at least one pair of spaced-apart electrodes, each of the plurality of electrical arcs being produced in response to a voltage pulse provided by the voltage pulse generator, and wherein the IVL system is further configured to generate at least one non-arcing voltage pulse.

Clause 2. The IVL catheter of clause 1, wherein the at least one non-arcing voltage pulse is configured to follow the produced plurality of electrical arcs.

Clause 3. The IVL catheter of clause 1 and/or clause 2, wherein the at least one non-arcing voltage pulse comprises a voltage magnitude of about 1000 volts or less.

Clause 4. The IVL catheter of clause 1 and/or clause 2, wherein the at least one non-arcing voltage pulse comprises a voltage magnitude of about 1,200 volts or less.

Clause 5. The IVL catheter of one or more of clauses 1-4, wherein the at least one non-arcing voltage pulse does not produce a visible electrical arc between the spaced-apart electrodes.

Clause 6. The IVL catheter of one or more of clauses 1-5, wherein the voltage pulse generator comprises a capacitor bank storing energy for generating voltage pulses that comprises residual energy after the production of electrical arcs, and wherein the non-arcing voltage pulse comprises a release of the residual energy from the capacitor bank.

Clause 7. The IVL catheter of clause 6, wherein the release of residual energy is automatically executed after a predetermined number of electrical arcs are produced.

Clause 8. The IVL catheter of one or more of clauses 1-5, further comprising a residual energy threshold, wherein the controller is configured to compare a residual energy stored in the voltage pulse generator with the residual energy threshold and to determine if the residual energy is above or below the residual energy threshold.

Clause 9. The IVL catheter of clause 8, wherein the controller is configured to release residual energy from the voltage pulse generator if the residual energy is determined by the controller to be above the residual energy threshold.

Clause 10. The IVL catheter of one or more of clauses 8-9, wherein the controller is configured to issue an alert if the residual energy is determined to be above the residual energy threshold.

Clause 11. The IVL catheter of clause 10, wherein the alert comprises a visual, auditory or haptic alert.

Clause 12. The IVL catheter of one or more of clauses 8-11, further comprising an EPROM in operative association and communication with the controller, wherein the EPROM comprises the residual energy threshold.

Clause 13. The IVL catheter of one or more of clauses 1-11, further comprising an EPROM in operative association and communication with the controller.

Clause 14. The IVL catheter of one of more of clauses 1-13, wherein the controller is configured to count the non-arcing voltage pulses.

Clause 15. The IVL catheter of clause 14, wherein the controller is further configured to determine if the counted non-arcing voltage pulses has met a predetermined threshold number of non-arcing voltage pulses.

Clause 16. The IVL catheter of clause 15, wherein the controller is further configured to lock out or prevent any further production of electrical arcs if the number of counted non-arcing voltage pulses equals the predetermined threshold number of non-arcing voltage pulses.

Clause 17. The IVL catheter of clause 14, wherein the controller is further configured to count the number of electrical arcs produced based on at least the number of generated non-arcing voltage pulses.

Clause 18. The IVL catheter of clause 1 wherein the controller is configured to determine an impedance of the IVL catheter.

Clause 19. The IVL catheter of clause 18 further comprising at least one of a voltage monitor or a current monitor and wherein the controller is configured to determine an impedance of the IVL catheter using voltage or current information determined using at least one of the voltage or current monitor.

Clause 20. The IVL catheter of clause 18 wherein IVL catheter stores a predetermined impedance threshold and wherein the controller is configured to compare the determined impedance with the impedance threshold and to determine if the determined impedance is above or below the impedance threshold.

Clause 21. The IVL catheter of clause 20, wherein the controller is configured to alert an operator of the determined impedance and/or whether the determined impedance is above or below the impedance threshold.

Clause 22. The IVL catheter of clause 21, wherein the controller is configured to instruct the voltage pulse generator to adjust the voltage magnitude in response to a determination that the impedance is above the impedance threshold.

Clause 23. The IVL catheter of clause 21, wherein the controller is configured to increase the voltage magnitude.

Clause 24. The IVL catheter of one or more of clauses 20-23, wherein the controller is configured to instruct the voltage pulse generator to adjust the duration of the voltage pulse that results in electrical arcs in response to a determination that the impedance is above the impedance threshold.

Clause 25. The IVL catheter of clause 18, wherein the controller is configured to increase the duration of the voltage pulses that result in electrical arcs to adapt to changing impedance.

Clause 26. A method for executing an intravascular lithotripsy (“IVL”) procedure, comprising: providing the IVL catheter of one or more of clauses 1-10; instructing the generation of a plurality of voltage pulses configured to produce electrical arcs between the spaced-apart electrodes; and instructing the generation of at least one non-arcing voltage pulse following the produced electrical arcs.

Clause 27. The method of clause 26, further comprising generating the at least one non-arcing voltage pulse by closing gates and/or relays within the voltage pulse generator.

Clause 28. The method of one or more of clauses 26 and 27, further comprising: monitoring or measuring the residual energy in the capacitor bank; establishing a predetermined residual energy threshold; determining if the monitored or measured residual energy is above or below the predetermined residual energy threshold; and if the determined residual energy is above the predetermined residual energy threshold, releasing at least some of the residual energy from the voltage pulse generator.

Clause 29. The method of clause 26, further comprising executing a non-arcing voltage pulse to release at least some residual energy from the voltage pulse generator.

Clause 30. The method of clause 26, further comprising closing gates and/or relays within a capacitor bank to release at least some of the residual energy from the capacitor bank.

Clause 31. The method of one or more of clauses 26-30, further comprising measuring one or both of current and/or voltage during the non-arcing voltage pulse; determining an impedance of the IVL catheter based on the measured current and/or voltage; comparing the determined impedance with an established impedance threshold value; and if the compared determined impedance is above the established impedance threshold value, adjusting the magnitude of voltage and/or duration of voltage pulses in a subsequent series of two or more voltage pulses, wherein each of the two or more voltage pulses are configured to generate electrical arcs between the spaced-apart electrodes.

Clause 32. An IVL catheter system, device and method as shown and described.

Further, the methods may be practiced by a computer system including one or more processors and computer-readable media such as computer memory. In particular, the computer memory may store computer-executable instructions that when executed by one or more processors cause various functions to be performed, such as the acts recited in the embodiments.

Embodiments of the present invention may comprise or utilize a special purpose or general-purpose computer including computer hardware, as discussed in greater detail below. Embodiments within the scope of the present invention also include physical and other computer-readable media for carrying or storing computer-executable instructions and/or data structures. Such computer-readable media can be any available media that can be accessed by a general purpose or special purpose computer system. Computer-readable media that store computer-executable instructions are physical storage media. Computer-readable media that carry computer-executable instructions are transmission media. Thus, by way of example, and not limitation, embodiments of the invention can comprise at least two distinctly different kinds of computer-readable media: physical computer-readable storage media and transmission computer-readable media.

Physical computer-readable storage media includes RAM, ROM, EEPROM, CD-ROM or other optical disk storage (such as CDs, DVDs, etc.), magnetic disk storage or other magnetic storage devices, or any other medium which can be used to store desired program code means in the form of computer-executable instructions or data structures and which can be accessed by a general purpose or special purpose computer.

A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer, the computer properly views the connection as a transmission medium. Transmissions media can include a network and/or data links which can be used to carry desired program code means in the form of computer-executable instructions or data structures and which can be accessed by a general purpose or special purpose computer. Combinations of the above are also included within the scope of computer-readable media.

Further, upon reaching various computer system components, program code means in the form of computer-executable instructions or data structures can be transferred automatically from transmission computer-readable media to physical computer-readable storage media (or vice versa). For example, computer-executable instructions or data structures received over a network or data link can be buffered in RAM within a network interface module (e.g., a “NIC”), and then eventually transferred to computer system RAM and/or to less volatile computer-readable physical storage media at a computer system. Thus, computer-readable physical storage media can be included in computer system components that also (or even primarily) utilize transmission media.

Computer-executable instructions comprise, for example, instructions and data which cause a general purpose computer, special purpose computer, or special purpose processing device to perform a certain function or group of functions. The computer-executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Those skilled in the art will appreciate that the invention may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, pagers, routers, switches, and the like. The invention may also be practiced in distributed system environments where local and remote computer systems, which are linked (either by hardwired data links, wireless data links, or by a combination of hardwired and wireless data links) through a network, both perform tasks. In a distributed system environment, program modules may be located in both local and remote memory storage devices.

Alternatively, or in addition, the functionality described herein can be performed, at least in part, by one or more hardware logic components. For example, and without limitation, illustrative types of hardware logic components that can be used include Field-programmable Gate Arrays (FPGAs), Application-specific Integrated Circuits (ASICs), Application-specific Standard Products (ASSPs), System-on-a-chip systems (SOCs), Complex Programmable Logic Devices (CPLDs), etc.

The present invention may be embodied in other specific forms without departing from its characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. An intravascular lithotripsy (“IVL”) catheter with improved efficiency,
comprising:
an elongate member;
a fluid-fillable enclosure formed of a material and surrounding a distal region of the elongate member;
at least one pair of spaced-apart electrodes operatively associated with the elongate member and within the fluid-fillable enclosure, wherein each pair of the at least one pair of spaced-apart electrodes form a spark gap;
a voltage pulse generator in operative electrical communication and association with the at least one pair of spaced-apart electrodes; and
a controller in operative association with the voltage pulse generator and configured to control the voltage pulse generator,
wherein the IVL system is configured to produce a plurality of electrical arcs across the at least one pair of spaced-apart electrodes, each of the plurality of electrical arcs being produced in response to a voltage pulse provided by the voltage pulse generator, and
wherein the IVL system is further configured to generate at least one non-arcing voltage pulse.

2. The IVL catheter of claim 1, wherein the at least one non-arcing voltage pulse is configured to follow the produced plurality of electrical arcs.

3. The IVL catheter of claim 1, wherein the at least one non-arcing voltage pulse comprises a voltage magnitude of about 1000 volts or less.

4. The IVL catheter of claim 1, wherein the at least one non-arcing voltage pulse comprises a voltage magnitude of about 1,200 volts or less.

5. The IVL catheter of claim 1, wherein the at least one non-arcing voltage pulse does not produce a visible electrical arc between the spaced-apart electrodes.

6. The IVL catheter of claim 1, wherein the voltage pulse generator comprises a capacitor bank storing energy for generating voltage pulses that comprises residual energy after the production of electrical arcs, and wherein the non-arcing voltage pulse comprises a release of the residual energy from the capacitor bank.

7. The IVL catheter of claim 6, wherein the release of residual energy is automatically executed after a predetermined number of electrical arcs are produced.

8. The IVL catheter of claim 1, further comprising a residual energy threshold, wherein the controller is configured to compare a residual energy stored in the voltage pulse generator with the residual energy threshold and to determine if the residual energy is above or below the residual energy threshold.

9. The IVL catheter of claim 8, wherein the controller is configured to release residual energy from the voltage pulse generator if the residual energy is determined by the controller to be above the residual energy threshold.

10. The IVL catheter of claim 8, wherein the controller is configured to issue an alert if the residual energy is determined to be above the residual energy threshold.

11. The IVL catheter of claim 10, wherein the alert comprises a visual, auditory or haptic alert.

12. The IVL catheter of claim 8, further comprising an EPROM in operative association and communication with the controller, wherein the EPROM comprises the residual energy threshold.

13. The IVL catheter of 1, further comprising an EPROM in operative association and communication with the controller.

14. The IVL catheter of one claim 1, wherein the controller is configured to count the non-arcing voltage pulses.

15. The IVL catheter of claim 14, wherein the controller is further configured to determine if the counted non-arcing voltage pulses has met a predetermined threshold number of non-arcing voltage pulses.

16. The IVL catheter of claim 15, wherein the controller is further configured to lock out or prevent any further production of electrical arcs if the number of counted non-arcing voltage pulses equals the predetermined threshold number of non-arcing voltage pulses.

17. The IVL catheter of claim 14, wherein the controller is further configured to count the number of electrical arcs produced based on at least the number of generated non-arcing voltage pulses.

18. The IVL catheter of claim 1 wherein the controller is configured to determine an impedance of the IVL catheter.

19. The IVL catheter of claim 18 further comprising at least one of a voltage monitor or a current monitor and wherein the controller is configured to determine an impedance of the IVL catheter using voltage or current information determined using at least one of the voltage or current monitor.

20. The IVL catheter of claim 18 wherein IVL catheter stores a predetermined impedance threshold and wherein the controller is configured to compare the determined impedance with the impedance threshold and to determine if the determined impedance is above or below the impedance threshold.

21. The IVL catheter of claim 20, wherein the controller is configured to alert an operator of the determined impedance and/or whether the determined impedance is above or below the impedance threshold.

22. The IVL catheter of claim 21, wherein the controller is configured to instruct the voltage pulse generator to adjust the voltage magnitude in response to a determination that the impedance is above the impedance threshold.

23. The IVL catheter of claim 21, wherein the controller is configured to increase the voltage magnitude.

24. The IVL catheter of claim 20, wherein the controller is configured to instruct the voltage pulse generator to adjust the duration of the voltage pulse that result in electrical arcs in response to a determination that the impedance is above the impedance threshold.

25. The IVL catheter of claim 18, wherein the controller is configured to increase the duration of the voltage pulses that result in electrical arcs to adapt to changing impedance.

26. A method for executing an intravascular lithotripsy (“IVL”) procedure, comprising:
providing the IVL catheter of claim 1;
instructing the generation of a plurality of voltage pulses configured to produce electrical arcs between the spaced-apart electrodes; and
instructing the generation of at least one non-arcing voltage pulse following the produced electrical arcs.

27. The method of claim 26, further comprising generating at least one non-arcing voltage pulse by closing of gates and/or relays within the voltage pulse generator.

28. The method of claim 26, further comprising:
monitoring or measuring the residual energy in the capacitor bank;
establishing a predetermined residual energy threshold;
determining if the monitored or measured residual energy is above or below the predetermined residual energy threshold; and
if the determined residual energy is above the predetermined residual energy threshold, releasing at least some of the residual energy from the voltage pulse generator.

29. The method of claim 26, further comprising executing a non-arcing voltage pulse to release at least some residual energy from the voltage pulse generator.

30. The method of claim 26, further comprising closing gates and/or relays within a capacitor bank to release at least some of the residual energy from the capacitor bank.

31. The method of claim 26, further comprising
measuring one or both of current and/or voltage during the non-arcing voltage pulse;
determining an impedance of the IVL catheter based on the measured current and/or voltage;
comparing the determined impedance with an established impedance threshold value; and
if the compared determined impedance is above the established impedance threshold value,
adjusting the magnitude of voltage and/or duration of voltage pulses in a subsequent series of two or more voltage pulses, wherein each of the two or more voltage pulses are configured to generate electrical arcs between the spaced-apart electrodes.
